(19) United States
(12) Patent Application Publication
(10) Pub. No.: US 2003/0138834 A1
(43) Pub. Date: Jul. 24, 2003

(54) Method for determining polynucleotide sequence variations

(76) Inventor: Elliott P. Dawson, Murfreesboro, TN (US)

Correspondence Address: David A. Farah, M.D., SHELDON & MAK PC, 9th Floor, 225 South Lake Avenue, Pasadena, CA, 91101, US

(21) Appl. No.: 10/346,156

(22) Filed: Jan. 15, 2003

Related U.S. Application Data
(63) Continuation-in-part of application No. 09/994,119, filed on Nov. 26, 2001, now abandoned.
Application No. 09/994,119 is a continuation of application No. 09/719,130, filed on Dec. 8, 2000, now Pat. No. 6,322,988.
Application No. 09/719,130 is a 371 of international application No. PCT/US99/18965, filed on Aug. 19, 1999.
(60) Provisional application No. 60/097,136, filed on Aug. 19, 1998.

(51) Int. Cl. C12Q 1/68; C12P 19/34

(52) U.S. Cl. 435/6; 435/91.2

(57) ABSTRACT

A method for determining the presence, location or identity, or a combination of these, of the nucleotides in a polynucleotide. A method for determining the presence, location or identity, or a combination of these, of one or more than one nucleotide difference between a first polynucleotide and a second polynucleotide, or between more than two polynucleotides.

### CROSS-REFERENCE TO RELATED APPLICATION

This Application is a continuation-in-part of U.S. patent application Ser. No. 09/994,119, filed Nov. 26, 2001 and titled “Method for Determining Polynucleotide Sequence Variations,” which is a continuation of U.S. patent application Ser. No. 09/719,130, filed Dec. 8, 2000 and titled “Method for Determining Polynucleotide Sequence Variations,” which is a national phase filing of PCT Application PCT/US99/18965 filed Aug. 19, 1999 and titled “Method for determining Polynucleotide Sequence Variations,” which claims the benefit of U.S. provisional patent application No. 60/097,136, filed Aug. 19, 1998 and titled “Detection of Single Nucleotide Polymorphisms,” the contents of which are incorporated herein by reference in their entirety.

### BACKGROUND

Individual DNA sequence variations in the human genome are known to directly cause specific diseases or conditions, or to predispose certain individuals to specific diseases or conditions. Such variations also modulate the severity or progression of many diseases. Additionally, DNA sequences vary between populations. Therefore, determining DNA sequence variations in the human genome is useful for making accurate diagnoses, for finding suitable therapies, and for understanding the relationship between genome variations and environmental factors in the pathogenesis of diseases and prevalence of conditions.

There are several types of DNA sequence variations in the human genome. These variations include insertions, deletions and copy number differences of repeated sequences. The most common DNA sequence variations in the human genome, however, are single base pair substitutions. These are referred to as single nucleotide polymorphisms (SNPs) when the variant allele has a population frequency of at least 1%.

SNPs are particularly useful in studying the relationship between DNA sequence variations and human diseases and conditions because SNPs are stable, occur frequently and have lower mutation rates than other genome variations such as repeating sequences. In addition, methods for detecting SNPs are more amenable to being automated and used for large-scale studies than methods for detecting other, less common DNA sequence variations.

A number of methods have been developed which can locate or identify SNPs. These methods include dideoxy fingerprinting (ddF), fluorescently labeled ddF, denaturation fingerprinting (DnF1R and DnF2R), single-stranded conformation polymorphism analysis, denaturing gradient gel electrophoresis, heteroduplex analysis, RNase cleavage, chemical cleavage, hybridization sequencing using arrays and direct DNA sequencing.

The known methods for locating or identifying SNPs are associated with certain disadvantages. For example, some known methods do not identify the specific base changes or the precise location of these base changes within a sequence. Other known methods are not amenable to analyzing many samples simultaneously or to analyzing pooled samples. Further, other known methods require different analytical conditions for the detection of each variation. Additionally, some known methods cannot be used to quantify known SNPs in genotyping assays. Further, many known methods have excessive limitations in throughput.

Thus, there is a need for a new method to determine the presence and identity of a variation in a nucleotide sequence between a first polynucleotide and a second polynucleotide, including the presence of an SNP in the genome of a human individual. Preferably, the method could determine the presence and identity of a variation in a nucleotide sequence between a first polynucleotide and a second polynucleotide in a pooled sample. Additionally preferably, the method could determine whether two or more variations reside on the same or different alleles in an individual, and could be used to determine the frequency of occurrence of the variation in a population. Further preferably, the method could screen large numbers of samples at a time with a high degree of accuracy.

### SUMMARY

According to one embodiment of the present invention, there is provided a method for determining the presence, location or identity, or a combination of these, of the nucleotides in a first polynucleotide, or for determining the presence, location or identity, or a combination of these, of one or more than one nucleotide difference between a first polynucleotides and a second polynucleotide. The method comprises, a) providing a sample of the first polynucleotide, b) selecting a region of the first polynucleotide potentially containing the variation, c) subjecting the selected region to a template producing amplification reaction to produce a first plurality of double stranded polynucleotide templates which includes the selected region, d) selecting a region of the templates potentially containing the variation, e) producing a first family of labeled, linear polynucleotide fragments from both strands of the templates simultaneously by a fragment producing reaction including, i) a set of at least two primers comprising a first primer and a second primer, ii) at least four types of non-sequence-terminating nucleotides, comprising at least two different sets of two Watson-Crick-pairing nucleotides or nucleotide analogs, and iii) two types of sequence-terminating non-Watson-Crick-pairing nucleotides or nucleotide analogs, comprising a first terminator and a second terminator, where one or more than one of the non-sequence-terminating nucleotides or the sequence-terminating non-Watson-Crick-pairing nucleotides is a nucleotide analog, where the first primer and the second primer flank the selected region of the template strands, where the first primer has a first primer label and the second primer has a second primer label, where at least a portion of one of the types of non-sequence-terminating nucleotides is labeled with a first nucleotide label, where the first terminator is labeled with a first terminator label and the second terminator is labeled with a second terminator label, where each of the first primer label, the second primer label, the first nucleotide label, the first terminator label and the second terminator label are all distinguishable from each other, where each of the first family of fragments is terminated by either the first terminator or the second terminator at the 3′ end of the fragment, and where the first family of fragments includes one or more than one fragment terminating at each possible base, represented by either the first terminator or the second terminator, of that portion of the selected region of both template strands flanked by a primer, and f) determining the location and identity of the bases in the selected region of the first polynucleotide by detecting the first primer label, the second primer label, the first nucleotide label, the first terminator label and the second terminator label present in the fragments. In another embodiment, the method additionally comprises comparing the location and identity of the bases determined with the location and identity of bases from a second polynucleotide, thereby identifying the presence and identity of a variation in a nucleotide sequence between the selected region of the first polynucleotide and a corresponding region of the second polynucleotide, after determining the location and identity of the bases in the selected region of the first polynucleotide.

According to another embodiment of the present invention, the sequence of the corresponding region of the second polynucleotide is determined by, a) providing a sample of the second polynucleotide, b) selecting a region of the second polynucleotide which corresponds to the region of the first polynucleotide potentially containing the variation, c) subjecting the corresponding region of the second polynucleotide to a template producing amplification reaction to produce a second plurality of double stranded polynucleotide templates which includes the corresponding region, d) producing a second family of labeled, linear polynucleotide fragments from both strands of the template simultaneously by a fragment producing reaction including, i) a set of at least two primers comprising a third primer and a fourth primer, ii) at least four types of non-sequence-terminating nucleotides, comprising at least two different sets of two Watson-Crick-pairing nucleotides or nucleotide analogs, and iii) two types of sequence-terminating non-Watson-Crick-pairing nucleotides or nucleotide analogs, comprising a third terminator and a fourth terminator, where the third primer and the fourth primer flank the selected region of the template strands, where each of the second family of fragments is terminated by either the third terminator or the fourth terminator at the 3′ end of the fragment, and where the second family of fragments includes one or more than one fragment terminating at each possible base, represented by the either the third terminator or the fourth terminator, of that portion of the selected region of both template strands flanked by a primer, e) determining the location and identity of at least some of the bases in the corresponding region of the second polynucleotide.

In another embodiment, the location and identity of the bases of the corresponding region of the second polynucleotide is determined simultaneously with determining the location and identity of the bases in the selected region of the first polynucleotide. In another embodiment, producing the first family of labeled, linear polynucleotide fragments and producing the second family of labeled, linear polynucleotide fragments is performed in one reaction. In another embodiment, the third primer has a third primer label and the fourth primer has a fourth primer label, and where the third primer label and the fourth primer label are distinguishable from each other. In another embodiment, at least a portion of one of the types of non-sequence-terminating nucleotides in the production of the second family of labeled, linear polynucleotide fragments is labeled with a second nucleotide label. In another embodiment, the third terminator is labeled with a third terminator label and the fourth terminator is labeled with a fourth terminator label, and where the third terminator label and the fourth terminator label are distinguishable from each other. In another embodiment, the third primer has a third primer label, the fourth primer has a fourth primer label and at least a portion of one of the types of non-sequence-terminating nucleotides in the production of the second family of labeled, linear polynucleotide fragments is labeled with a second nucleotide label, and where the third primer label, the fourth primer label and the second nucleotide label are all distinguishable from each other. In another embodiment, the third primer has a third primer label, the fourth primer has a fourth primer label, the third terminator is labeled with a third terminator label and the fourth terminator is labeled with a fourth terminator label, and where the third primer label, the fourth primer label, the third terminator label and the fourth terminator label are all distinguishable from each other. In another embodiment, at least a portion of one of the types of non-sequence-terminating nucleotides in the production of the second family of labeled, linear polynucleotide fragments is labeled with a second nucleotide label, where the third terminator is labeled with a third terminator label and the fourth terminator is labeled with a fourth terminator label, and where the second nucleotide label, the third terminator label and the fourth terminator label are all distinguishable from each other. In another embodiment, the third primer has a third primer label, the fourth primer has a fourth primer label, at least a portion of one of the types of non-sequence-terminating nucleotides in the production of the second family of labeled, linear polynucleotide fragments is labeled with a second nucleotide label, the third terminator is labeled with a third terminator label and the fourth terminator is labeled with a fourth terminator label, and where the third primer label, the fourth primer label, the second nucleotide label, the third terminator label and the fourth terminator label are all distinguishable from each other.

According to another embodiment of the present invention, there is provided a method for determining the presence, location or identity, or a combination of these, of the nucleotides in a first polynucleotide, or for determining the presence, location or identity, or a combination of these, of one or more than one nucleotide difference between a first polynucleotides and a second polynucleotide, comprising: a) providing a sample of the first polynucleotide, b) selecting a region of the first polynucleotide potentially containing the variation, c) subjecting the selected region to a template producing amplification reaction to produce a first plurality of double stranded polynucleotide templates which includes the selected region, d) selecting a region of the templates potentially containing the variation, e) producing a first family of labeled, linear polynucleotide fragments from both strands of the templates simultaneously by a fragment producing reaction including, i) a set of at least two primers comprising a first primer and a second primer, ii) at least four types of non-sequence-terminating nucleotides, comprising at least two different sets of two Watson-Crick-pairing nucleotides or nucleotide analogs, and iii) two types of sequence-terminating non-Watson-Crick-pairing nucleotides or nucleotide analogs, comprising a first terminator and a second terminator, where one or more than one of the non-sequence-terminating nucleotides or the sequence-terminating non-Watson-Crick-pairing nucleotides is a nucleotide analog, where the first primer and the second primer flank the selected region of the template strands, where each of the first family of fragments is terminated by either the first terminator or the second terminator at the 3′ end of the fragment, and where the first family of fragments includes one or more than one fragment terminating at each possible base, represented by the either the first terminator or the second terminator, of that portion of the selected region of both template strands flanked by a primer, and f) determining the location and identity of the bases in the selected region. In another embodiment, the method additionally comprises comparing the location and identity of the bases determined with the location and identity of bases from a second polynucleotide, thereby identifying the presence and identity of a variation in a nucleotide sequence between the selected region of the first polynucleotide and a corresponding region of the second polynucleotide, after determining the location and identity of the bases in the selected region of the first polynucleotide. In another embodiment, the first primer has a first primer label and the second primer has a second primer label, and where the first primer label and the second primer label are distinguishable from each other. In another embodiment, at least a portion of one of the types of non-sequence-terminating nucleotides is labeled with a first nucleotide label. In another embodiment, the first terminator is labeled with a first terminator label and the second terminator is labeled with a second terminator label, and where the first terminator label and the second terminator label are distinguishable from each other. In another embodiment, the first primer has a first primer label, the second primer has a second primer label and at least a portion of one of the types of non-sequence-terminating nucleotides is labeled with a first nucleotide label, and where the first primer label, the second primer label and the first nucleotide label are all distinguishable from each other. In another embodiment, the first primer has a first primer label, the second primer has a second primer label, the first terminator is labeled with a first terminator label and the second terminator is labeled with a second terminator label, and where the first primer label, the second primer label, the first terminator label and the second terminator label are all distinguishable from each other. In another embodiment, at least a portion of one of the types of non-sequence-terminating nucleotides is labeled with a first nucleotide label, where the first terminator is labeled with a first terminator label and the second terminator is labeled with a second terminator label, and where the first nucleotide label, the first terminator label and the second terminator label are all distinguishable from each other. In another embodiment, the first primer has a first primer label, the second primer has a second primer label, at least a portion of one of the types of non-sequence-terminating nucleotides is labeled with a first nucleotide label, the first terminator is labeled with a first terminator label and the second terminator is labeled with a second terminator label, and where the first primer label, the second primer label, the first nucleotide label, the first terminator label and the second terminator label are all distinguishable from each other.

In another embodiment, the sequence of the corresponding region of the second polynucleotide is determined by: a) providing a sample of the second polynucleotide, b) selecting a region of the second polynucleotide which corresponds to the region of the first polynucleotide potentially containing the variation, c) subjecting the corresponding region of the second polynucleotide to a template producing amplification reaction to produce a second plurality of double stranded polynucleotide templates which includes the corresponding region, d) producing a second family of labeled, linear polynucleotide fragments from both strands of the template simultaneously by a fragment producing reaction including, i) a set of at least two primers comprising a third primer and a fourth primer, ii) at least four types of non-sequence-terminating nucleotides, comprising at least two different sets of two Watson-Crick-pairing nucleotides or nucleotide analogs, and iii) two types of sequence-terminating non-Watson-Crick-pairing nucleotides or nucleotide analogs, comprising a third terminator and a fourth terminator, where the third primer and the fourth primer flank the selected region of the template strands, where each of the second family of fragments is terminated by either the third terminator or the fourth terminator at the 3′ end of the fragment, and where the second family of fragments includes one or more than one fragment terminating at each possible base, represented by the either the third terminator or the fourth terminator, of that portion of the selected region of both template strands flanked by a primer, e) determining the location and identity of at least some of the bases in the corresponding region of the second polynucleotide. In another embodiment, the location and identity of the bases of the corresponding region of the second polynucleotide is determined simultaneously with determining the location and identity of the bases in the selected region of the first polynucleotide. In another embodiment, producing the first family of labeled, linear polynucleotide fragments and producing the second family of labeled, linear polynucleotide fragments is performed in one reaction. In another embodiment, the third primer has a third primer label and the fourth primer has a fourth primer label, and where the third primer label and the fourth primer label are distinguishable from each other. In another embodiment, at least a portion of one of the types of non-sequence-terminating nucleotides in the production of the second family of labeled, linear polynucleotide fragments is labeled with a second nucleotide label. In another embodiment, the third terminator is labeled with a third terminator label and the fourth terminator is labeled with a fourth terminator label, and where the third terminator label and the fourth terminator label are distinguishable from each other. In another embodiment, the third primer has a third primer label, the fourth primer has a fourth primer label and at least a portion of one of the types of non-sequence-terminating nucleotides in the production of the second family of labeled, linear polynucleotide fragments is labeled with a second nucleotide label, and where the third primer label, the fourth primer label and the second nucleotide label are all distinguishable from each other. In another embodiment, the third primer has a third primer label, the fourth primer has a fourth primer label, the third terminator is labeled with a third terminator label and the fourth terminator is labeled with a fourth terminator label, and where the third primer label, the fourth primer label, the third terminator label and the fourth terminator label are all distinguishable from each other. In another embodiment, at least a portion of one of the types of non-sequence-terminating nucleotides in the production of the second family of labeled, linear polynucleotide fragments is labeled with a second nucleotide label, where the third terminator is labeled with a third terminator label and the fourth terminator is labeled with a fourth terminator label, and where the second nucleotide label, the third terminator label and the fourth terminator label are all distinguishable from each other. In another embodiment, the first primer has a first primer label, the second primer has a second primer label, the third primer has a third primer label, the second primer has a second primer label, at least a portion of one of the types of non-sequence-terminating nucleotides in the production of the first family of labeled, linear polynucleotide fragments is labeled with a first nucleotide label, at least a portion of one of the types of non-sequence-terminating nucleotides in the production of the second family of labeled, linear polynucleotide fragments is labeled with a second nucleotide label, the first terminator is labeled with a first terminator label, the second terminator is labeled with a second terminator label, the third terminator is labeled with a third terminator label and the fourth terminator is labeled with a fourth terminator label, and where the first primer label, the second primer label, the third primer label, the fourth primer label, the first nucleotide label, the second nucleotide label, the first terminator label, the second terminator label, the third terminator label and the fourth terminator label are all distinguishable from each other.

In another embodiment, the selected region of the first polynucleotide comprises a plurality of discontinuous sequences on the first polynucleotide. In another embodiment, the template producing amplification reaction comprises subjecting the selected region to PCR. In another embodiment, the template producing amplification reaction comprises subjecting the selected region to RT-PCR. In another embodiment, the first plurality of double stranded polynucleotide templates comprises double stranded nucleic acid strands of between about 50 and 50,000 nucleotides per strand. In another embodiment, the method further comprises purifying the temples to remove other amplification reaction components after subjecting the selected region to a template producing amplification reaction. In another embodiment, the fragment producing amplification reaction comprises subjecting the selected region to PCR. In another embodiment, the fragment producing amplification reaction comprises subjecting the selected region to RT-PCR. In another embodiment, the selected region of the template strands is between about 100 and 1000 nucleotides per strand. In another embodiment, the at least four types of non-sequence-terminating nucleotides comprise dATP, dCTP, dGTP and dTTP. In another embodiment, the at least four types of non-sequence-terminating nucleotides comprise dATP, dCTP, dGTP and dUTP. In another embodiment, one or more than one of the at least four types of the non-sequence-terminating nucleotides comprise an alpha thio dNTP analog. In another embodiment, two of the at least four types of the non-sequence-terminating nucleotides comprise an alpha thio dNTP analog, two of the at least four types of the non-sequence-terminating nucleotides comprise dNTPs, and the two sequence-terminating non-Watson-Crick-pairing nucleotides comprise ddNTPs corresponding to the two alpha thio phosphate dNTPs. In another embodiment, the two alpha thio phosphate non-sequence-terminating nucleotides are present in an initial concentration of between about 10% and 50% of that of the initial concentration of the two dNTP non-sequence-terminating nucleotides. In another embodiment, one or more than one of the two types of the sequence-terminating non-Watson-Crick-pairing nucleotides comprises an alpha thio dNTP analog. In another embodiment, one or more than one of the two types of the sequence-terminating non-Watson-Crick-pairing nucleotides is a 2′ deoxnucleotide triphosphates analog having an extension blocking moiety at the 3′ position. In another embodiment, the extension blocking moiety is selected from the group consisting of an azide moiety, an amino moiety, a deoxy moiety, a fluoro moiety and a methoxy moiety. In another embodiment, one or more than one of the sequence-terminating non-Watson-Crick-pairing nucleotides has an acyclo analog of a nucleotide sugar moiety. In another embodiment, the first terminator comprises a pyrimidine nucleotide and where the second terminator comprises a purine nucleotide. In another embodiment, the first terminator and the second terminator are selected from the group consisting of ddATP:ddCTP, ddATP:ddGTP, ddCTP:ddTTP, ddGTP:ddTTP, ddCTP:ddUTP, ddGTP:ddUTP and one of the foregoing pairs where one or both members of the pair is a nucleotide analog. In another embodiment, one or more than one of the first primer label, the second primer label, the first nucleotide label, the first terminator label and the second terminator label are selected from the group consisting of fluorescent labels, fluorescent energy transfer labels, luminescent labels, chemiluminescent labels, phosphorescent labels and photoluminescent labels. In another embodiment, the portion of one of the types of non-sequence-terminating nucleotides that is labeled with a first nucleotide label comprises between about 1% and about 10% of the total concentration of unlabeled non-sequence-terminating nucleotides. In another embodiment, the method further comprises purifying the labeled reaction products from the fragment producing reaction before determining the location and identity of the bases in the selected region of the first polynucleotide. In another embodiment, one or more of the first primer, the second primer, a portion of one of the types of non-sequence-terminating nucleotides, the first terminator and the second terminator is labeled, and where determining the location and identity of the bases in the selected region of the first polynucleotide is accomplished by detecting the label or labels.

### DESCRIPTION

The present invention includes a method for determining the presence, location or identity, or a combination of these, of the nucleotides in a polynucleotide. The present invention also a method for determining the presence, location or identity, or a combination of these, of one or more than one nucleotide difference between a first polynucleotide and a second polynucleotide, or between more than two polynucleotides. Among other uses, the present method can locate and identify single nucleotide polymorphisms present in the human genome. Further, the present method can discover previously unidentified genome variations between individuals, between an individual and a population, and between populations. Also, the present method can determine the frequency or distribution of genome variations within populations. Additionally, the present method can relate specific genome variations found in a population to specific phenotypes within that population. Further, the present method can determine the allelic distribution of genome variations in individuals and populations.

More specifically, the present method of the present invention can provide the following types of information on polynucleotide sequence variation between two polynucleotides. First, the present method can identify the position of all the nucleotides in a selected region of a first polynucleotide that are different from one or more than one additional polynucleotides. Second, the present method can identify which nucleotide has replaced another nucleotide in a polynucleotide. Third, the present method can determine the proportion of the polynucleotide molecules that have each of the nucleotide changes that can occur at a given location in the sequence. Fourth, where two different polynucleotides have a plurality of nucleotide differences, the present method can provide information on which differences occur together.

The present method has several combined advantages over known methods. Generally, the present method provides more types of information, is more widely applicable and is simpler to perform. Particularly advantageous, the present method is a single technology that can simultaneously identify and quantitate known and unknown variations and determine the locations, identities and frequencies of all variations between two populations of polynucleotides. Additionally, the present method can determine whether two or more genetic variations reside on the same or different alleles in an individual, and can be used to determine the frequency of occurrence of the variation in a population.

Further, the present method can be used on any type of polynucleotide, from any source. In addition to determining the location and identity of SNPs, the present method can be used to determine the presence and type of polynucleotide variations including substitutions, deletions, insertions, expansions and contractions involving multiple nucleotides, and truncated or chimeric molecules. Further, the present method can identify alterations in the relative copy number of sequences in diploid organisms that involve the loss of one copy of a polynucleotide such as loss of heterozygosity, or that involve the gain of additional copies of a polynucleotide such as conditions in which extra copies of chromosomes, genes or gene segments are present.

Additionally, in population studies, the present method can be used to determine the frequencies of each polynucleotide variation by analysis of a single pooled sample that is composed of samples taken from multiple individuals. Finally, the present method can be used to estimate the proportion of the population that is susceptible or resistant to a factor that is dependant on the presence or absence of a particular polynucleotide variation or to detect polynucleotide variations in populations that occur over time, such as in cultures of pooled bacteria. Also, the present method can be automated.

As used herein, the term “nucleotide” is understood to include a nucleotide triphosphate.

The present method preferably comprises providing a sample of a first polynucleotide. Then, one or more than one specific regions of the first polynucleotide are selected where the presence, location or identity of at least one sequence variation is to be determined. Next, the selected region is subjected to a template producing amplification reaction. In a preferred embodiment, the templates produced are purified to remove other amplification reaction components.

Then, a family of labeled, linear polynucleotide fragments is produced from both strands of the template simultaneously by a fragment producing reaction using a set of primers. The family of fragments produced by this reaction includes fragments which terminate by a sequence-terminating non-Watson-Crick-pairing nucleotide at the 3′ end at each possible base, represented by the sequence-terminating non-Watson-Crick-pairing nucleotide, of both templates strands flanked by the primers.

Finally, the location and identity of each base in the selected region of the template from the first polynucleotide are identified using the labels present in the fragments. The location and identity are compared to a known reference sequence, or are compared with corresponding information determined from a family of labeled, linear polynucleotide fragments produced from a second polynucleotide using the present method. The comparison yields information about the presence, location or identity of one or more than one sequence difference between the first polynucleotide and the reference sequence, or between the first polynucleotide and the second polynucleotide. The present method will now be discussed in greater detail.

1) Provision of Sample Polynucleotide:

Before template amplification, the polynucleotide or polynucleotides of interest must be obtained in suitable quantity and quality for the chosen amplification method to be used. Some suitable samples can be purchased from suppliers such as the American Type Culture Collection, Rockville, Md. US or Coriell Institute for Medical Research, Camden, N.J. US. Additionally, commercially available kits for obtaining suitable polynucleotide samples from various sources are available from Qiagen Inc., Chatsworth, Calif. US; Invitrogen Corporation, Carlsbad, Calif. US; Promega Corporation, Madison, Wis. US, among other suppliers. Further, general methods for obtaining polynucleotides from various sources for amplification methods including PCR and RT-PCR are well known to those with skill in the art.

Advantageously, the present method allows for simultaneous analysis of polynucleotides obtained from a plurality of samples. If two or more polynucleotide samples are pooled prior to analysis, then the polynucleotide samples are preferably mixed in equal proportions.

2) Selection of One or More than One Region of the Polynucleotide for Analysis:

Next, one or more than one specific regions of a first polynucleotide are selected where the presence, location or identity of at least one sequence variation is to be determined. As used in this disclosure, “region” should be understood to include a plurality of discontinuous sequences on the same polynucleotide. Region selection can be based upon known sequence information for the same or related polynucleotides, or can be based upon the region of interest of a reference polynucleotide which is sequenced using techniques well known to those with skill in the art.

3) Amplification of the Selected Region:

Once the region is selected, the region is subjected to an amplification reaction according to techniques known to those with skill in the art, to produce templates. As used in this disclosure, “template” or “templates” should be understood to include a plurality of templates produced from discontinuous sequences on the same polynucleotide. In a preferred embodiment, the templates produced by this amplification reaction comprise double stranded nucleic acid strands of between about 50 and 50,000 nucleotides per strand. In a particularly preferred embodiment, the amplification method is PCR where the polynucleotide being analyzed is DNA, or is RT-PCR where the polynucleotide being analyzed is RNA, though the templates can be produced by any suitable amplification method for the polynucleotide being analyzed as will be understood by those with skill in the art with reference to this disclosure. Suitable kits for performing PCR and RT-PCR are available from a number of commercial suppliers, including Amersham Pharmacia Biotech, Inc., Piscataway, N.J. US; Invitrogen Corporation; and Perkin-Elmer, Corp., Norwalk, Conn. US, among other sources.

4) Template Purification:

In a preferred embodiment, the templates produced by the amplification reaction are purified from other amplification reaction components according to techniques known to those with skill in the art. For example, the amplification reaction mixture can be subjected to polyacrylamide gel electrophoresis or agarose gel electrophoresis, and templates having the expected size are purified from the other amplification reaction components by ethanol or isopropanol precipitation, membrane purification or column purification. After purification, the templates should be kept in solution, preferably in sterile, nuclease free, 18 megaohm water or in 0.1×TE.

5) Production of a Family of Labeled, Linear Polynucleotide Fragments:

The templates produced by amplification are then used to produce a family of labeled, linear polynucleotide fragments from both strands of each template simultaneously by a fragment producing reaction using a set of primers. The fragment producing reaction is similar to an amplification reaction except that the polynucleotide fragments amplified comprise a family of fragments from both template strands flanked by the primers, and the family of fragments terminate by a sequence-terminating non-Watson-Crick-pairing nucleotide at the 3′ end, and terminate at each possible base corresponding to a sequence-terminating non-Watson-Crick-pairing nucleotide, rather than a single polynucleotide sequence spanning the full length of the template strands flanked by the primers.

In a preferred embodiment, the fragment producing reaction is performed as follows, though other equivalent procedures are also suitable as will be understood by those with skill in the art with reference to this disclosure. First, a region of the polynucleotide sequence lying within the template is selected for analysis. Next, a pair of primers is synthesized that flanks the selected region. In a preferred embodiment, the polynucleotide length between the forward and reverse primer pair from their respective 3′ ends is between about 50 and 2000 nucleotides in length. In a particularly preferred embodiment, the polynucleotide length between the forward and reverse primer pair from their respective 3′ ends is between about 100 and 1000 nucleotides in length.

Then, a reaction mixture is made comprising the template, a solvent, the primer pair, a set of four ‘non-sequence-terminating nucleotides,’ a pair of ‘sequence-terminating non-Watson-Crick-pairing nucleotides,’ buffer, a divalent cation, DNA dependant DNA polymerase and one or more than one detectible labeling agent. These components of the reaction mixture will now be discussed in detail.

The reaction mixture comprises between about 1 pg and 200 ng, and more preferably between about 100 and 150 ng, of the template placed in a volume of solvent comprising between about 1 and 3 μl of sterile, nuclease free, 18 megaohm water or 0.1×TE buffer. The synthesized primer pair is added to this reaction mixture in a final concentration of between about 1 and 50 pMoles per reaction for a total reaction volume of about 20 μl.

The reaction mixture further comprises four non-sequence-terminating nucleotides, consisting of two pairs of complementary nucleotides. In a preferred embodiment, the four non-sequence-terminating nucleotides are dATP, dCTP, dGTP and dTTP (2′-deoxy-Thymidine -5′-o-triphosphate). However, dUTP can advantageously be used in place of dTTP to improve results, such as when there are more than five contiguous thymine residues in the template to be analyzed. Further, a mixture of both dTTP and dUTP can be used at the same time. In a preferred embodiment, one or more than one of the four non-sequence-terminating nucleotides is an alpha thio dNTP analog having an alpha thio phosphate as a portion of the triphosphate ester groups. Use of an alpha thio dNTP analog as one or more than one of the four non-sequence-terminating nucleotides has been found to advantageously improve resolution during chromatography and to decrease the rate of artifacts when using a DNA polymerase having 3′ exonuclease activity. Suitable thio modified nucleotide triphosphates are available from a number of commercial suppliers, including Sigma-Aldrich, Saint Louis, Mo. US; Amersham Pharmacia Biotech; Trilink Biotechnologies, San Diego, Calif. US, among other sources.

In a preferred embodiment, only one of the four non-sequence-terminating nucleotides has one alpha thio phosphate as a portion of its triphosphate ester groups. In another preferred embodiment, only two of the four non-sequence-terminating nucleotides have one alpha thio phosphate as a portion of their triphosphate ester groups. In another preferred embodiment, only three of the four non-sequence-terminating nucleotides have one alpha thio phosphate as a portion of their triphosphate ester groups.

In a particularly preferred embodiment, only two of the four non-sequence-terminating nucleotides have one alpha thio phosphate as a portion of their triphosphate ester groups, and these two alpha thio phosphate non-sequence-terminating nucleotides correspond to the two sequence-terminating non-Watson-Crick-pairing nucleotides, such as alpha thio phosphate dATP:dCTP when using sequence-terminating non-Watson-Crick-pairing nucleotides ddATP:ddCTP as the sequence-terminating non-Watson-Crick-pairing nucleotides; or alpha thio phosphate ddGTP:ddTTP when using ddGTP:ddTTP as the sequence-terminating non-Watson-Crick-pairing nucleotides. Such alpha thio phosphate non-sequence-terminating nucleotides corresponding to the sequence-terminating non-Watson-Crick-pairing nucleotides advantageously allows the rate of termination to be more favored than the non terminating extension reaction afforded when using dNTPs rather than the alpha thio phosphate dNTPs.

In a preferred embodiment, the reaction mixture initially comprises the four non-sequence-terminating nucleotides in approximately equimolar ratios to one another. In another preferred embodiment, the reaction mixture comprises each of the four non-sequence-terminating nucleotides in an initial concentration of between about 1 μmolar and 1 mmolar. In another preferred embodiment, the reaction mixture comprises each of the four non-sequence-terminating nucleotides in an initial concentration of between about 20 and 200 μmolar. In a preferred embodiment, the reaction mixture comprises two dNTPs and two alpha thio phosphate dNTPs as the non-sequence-terminating nucleotides, and two ddNTPs as the sequence-terminating non-Watson-Crick-pairing nucleotides corresponding to the two alpha thio phosphate dNTPs, where the two alpha thio phosphate non-sequence-terminating nucleotides are present initial concentration of between about 10% and 50% of that of the initial concentration of the two dNTPs.

The reaction mixture further comprises approximately equal concentrations of two sequence-terminating non-Watson-Crick-pairing nucleotides that can be any suitable pair of nucleotides that prevent extension of the polynucleotide after the sequence-terminating nucleotide is incorporated into a polynucleotide, as will be understood by those with skill in the art with reference to this disclosure. In a preferred embodiment, one of the two ddNTPs is a pyrimidine nucleotide and the other is a purine nucleotide.

In one embodiment, the pair of sequence-terminating non-Watson-Crick-pairing nucleotides are 2′ deoxnucleotide triphosphates having an extension blocking moiety at the 3′ position, such as for example, a moiety selected from the group consisting of an azide moiety, an amino moiety, a deoxy moiety, a fluoro moiety and a methoxy moiety. In a preferred embodiment, the sequence-terminating non-Watson-Crick-pairing nucleotides is an acyclo analog of the sugar moiety of the nucleotide, such as for example, acyclo adenosine triphosphate (acycloATP), acyclo Guanosine triphosphate, acyclo Thymidine triphosphate, acyclo Uridine triphosphate or acyclo Cytidine triphosphate (Perkin Elmer Life Sciences, Boston, Mass.). Additionally, the pair of sequence-terminating non-Watson-Crick-pairing nucleotides can be a pair of nucleotides, such as for example 3′ deoxynucleotide triphosphates, that substantially limit polynucleotide extension after the sequence terminator is incorporated into the polynucleotide, thereby functioning as sequence-terminating nucleotides for the purpose of this invention even though polynucleotide extension is not totally prevented on all growing polynucleotides, as will be understood by those with skill in the art with reference to this disclosure. In a preferred embodiment, one or more than one of the sequence-terminating non-Watson-Crick-pairing nucleotides is an alpha thio dNTP analog having an alpha thio phosphates as a portion of the triphosphate ester groups. Use of an alpha thio dNTP analog as one or more than one of the sequence-terminating non-Watson-Crick-pairing nucleotides has been found to advantageously improve resolution during chromatography and to decrease the rate of artifacts when using a DNA polymerase having 3′ exonuclease activity. Suitable thio modified nucleotide triphosphates are available from a number of commercial suppliers, including Amersham Pharmacia Biotech; Perkin Elmer; Sigma-Aldrich; Trilink Biotechnologies, among other sources. In a preferred embodiment, the pair of sequence-terminating non-Watson-Crick-pairing nucleotides comprises two non-Watson-Crick-pairing bases of the set of 2′-3′ dideoxynucleotide triphosphates (ddNTP), or corresponding analogs, consisting of ddATP, ddCTP, ddGTP and ddTTP (or ddUTP in place of ddTTP). Suitable pairs include ddATP:ddCTP, ddATP:ddGTP, ddCTP:ddTTP, ddGTP:ddTTP. In a particularly preferred embodiment, the ddNTPs pair is either ddATP:ddCTP or ddGTP:ddTTP, or their corresponding analogs, either pair of which will result in complete sequence information about the entire template sequence lying between the 3′ ends of the primers.

The initial concentration of the pairs of sequence-terminating non-Watson-Crick-pairing nucleotides in the reaction mixture depends upon the efficiencies of the sequence-terminating non-Watson-Crick-pairing nucleotides to be used as a substrate for the polymerase, as will be understood by those with skill in the art with reference to this disclosure. In a preferred embodiment, the reaction mixture initially comprises each of the two sequence-terminating non-Watson-Crick-pairing nucleotides in a concentration of between about 0.01 μM to 10 mM. In another preferred embodiment, the initial concentration of each of the two sequence-terminating non-Watson-Crick- pairing nucleotides is between about 10 μM and 500 μM. In a preferred embodiment, the reaction mixture initially comprises each of the two sequence-terminating non-Watson-Crick-pairing nucleotides in a concentration of 0.05% to about 10% of the initial concentration of the corresponding non-sequence-terminating nucleotide. However, the initial concentration of each sequence-terminating non-Watson-Crick-pairing nucleotide can be approximately equal to the initial concentration of the other sequence-terminating non-Watson-Crick-pairing nucleotide, or can be different from the initial concentration of the other sequence-terminating non-Watson-Crick-pairing nucleotide. Preferably, the concentration of each sequence-terminating non-Watson-Crick-pairing nucleotide will be optimized according to techniques known to those with skill in the art for reaction product length, signal strength and the respective efficiencies of the sequence-terminating non-Watson-Crick-pairing nucleotide as a substrate for the polymerases utilized.

The reaction mixture further comprises a buffer having sufficient buffering capacity to maintain the pH of the reaction mixture over a pH range of about 6.0 to 10.0 and over a temperature range of about 20° C. to 98° C. In a preferred embodiment, the buffer is Tris at a concentration of between about 10 mM and 500 mM, and preferably between about 50 mM and 300 mM.

The reaction mixture further comprises one or more than one divalent cation. In a preferred embodiment, the divalent cation is magnesium chloride salt in a concentration of between about 0.5 and 10 mM, and more preferably in a concentration of between about 1.5 and 3.0 mM. Manganese chloride salt in a concentration of between about 0.1 mM and 20 mM can also be used as appropriate.

The reaction mixture further comprises a polymerase, such as a DNA dependant DNA polymerase. The polymerase selected should preferably be thermostable, have minimal exonuclease, endonuclease or other DNA degradative activity, and should have good efficiency and fidelity for the incorporation of ddNTPs into the synthesizing DNA strands. A suitable concentration of polymerase is between about 0.1 and 100 units per reaction, and more preferably a concentration of between about 1 and 10 units per reaction. Suitable polymerases are commercially available from Amersham Pharmacia Biotech, Inc., Perkin-Elmer Corporation, and Promega Corporation, among other suppliers.

In a preferred embodiment, the reaction mixture further comprises additional substances to improve yield or efficiency, enhance polymerase stability, and to alleviate artifacts. For example, other non-sequence-terminating nucleotides or supplemental non-sequence-terminating nucleotides, such as deoxyinosine triphosphate (dITP) or 7-deaza GTP can be employed in a concentration of between about 0.1 mM and 20 mM in place of dGTP to alleviate compression, stutters or stops that can occur in the fragment producing reaction. Also, for example, detergents and reducing agents can be added to stabilize the polymerase. Additionally, organic solvents such as glycerol, dimethylformamide, formamide, acetontrile and isopropanol can be added to the reaction mixture to improve annealing stringency of the primers. When present, the organic solvents preferably have a concentration of between about 0.1% and 20% by volume.

In addition to the above discussed reaction mixture components, it is essential that the reaction products produced by the fragment producing reaction contain one or more than one detectible label by incorporation of labeled primers, labeled non-sequence-terminating nucleotides, or labeled sequence-terminating non-Watson-Crick-pairing nucleotides, or a combination of the foregoing, depending on the number and types of samples being analyzed, and whether the samples are from pooled sources, as will be understood with reference to this disclosure. Among the types of labels suitable for performing the present method are fluorescent labels, fluorescent energy transfer labels, luminescent labels, chemiluminescent labels, phosphorescent labels and photoluminescent labels, though other types of labels are suitable as long as the labels are compatible with this method, the detection of multiple labels permits the discrimination of the labels from one another, and the reaction products can be measured by the labels. In a preferred embodiment, the label is either a fluorescent label or a fluorescent energy transfer label.

A wide variety of fluorescent labels, such as fluorescent dyes, are suitable for use in this method. Suitable fluorescent labels suitable should be chemically stable for their incorporation into the labeled reagents, and should be resistant to degradation during performance of this method. Further, the fluorescent labels should have only nominal influence on the migration of the reaction products when the reaction products are being analyzed. Additionally, the fluorescent labels should have good quantum efficiency for excitation and emission, and the spectral separation between the excitation wavelength and the emission wavelength should be at least 10 nanometers where they are capable of being spectrally resolved from one another at their emission wavelength having a minimum of 5 nanometers between their respective emissions. The excitation wavelengths are preferably between about 260 nm and 2000 nm and the emission wavelengths are preferably between about 280 nm and 2500 nm. Further, the fluorescent labels should preferably be capable of being attached to the primers, dNTPs and ddNTPs.

Examples of suitable fluorescent labels are fluorescent compounds derived from the family of fluoresceine and its derivatives, rhodamine and its derivatives, Bodipy®(4,4-difluoro -4-bora-3a,4a-diaza-s-indacene) and its derivatives, cyanine and its derivatives, and Europium chelates. Suitable fluorescent dye labels are commercially available from Molecular Probes, Inc., Eugene, Oreg. US and Research Qrganics, Inc., Cleveland, Ohio US, among other sources. Similarly, suitable energy transfer pairs are commercially available, such as Big Dyes™ from Perkin-Elmer Corporation. Further, custom-made primers with attached energy transfer pairs can be obtained from Amersham Pharmacia Biotech, Inc., among other suppliers.

The primers used in the reaction mixture can be labeled at their 5′ ends or internally with one or more than one labels as long as the 3′OH groups of the primers remain exposed to allow the polymerase to function with the primer. While both forward and reverse primers can be labeled with identical labels, it is preferred that the forward and reverse primers are labeled with different labels that can be distinguished from each another.

Suitable labeled primers can be prepared by any of several methods, or can be purchased commercially, as will be understood by those with skill in the art with reference to this disclosure. For example, fluorescent phosphoramidites can be used either to label the 5′ end of the primers or to internally label the primers. The primary amines can be labeled using standard N-hydroxy succinimide esters or other species of the fluorescent dyes reactive with the primary amines can be introduced into the primers as the primers are synthesized. Further, other reactive species such as sulfhydryl groups can be introduced into the primers and conjugated to fluorescent dyes having appropriate reactivities. A typical concentration of dye labeled primers for use in this method would be between about 1 pMole and 50 pMoles for a 20 μl reaction volume.

The sequence-terminating non-Watson-Crick-pairing nucleotides used in the reaction mixture are labeled. The labeled sequence-terminating non-Watson-Crick-pairing nucleotides terminate polynucleotide strand synthesis in the fragment producing reaction, as well as allow identification of the base at which strand termination occurs in the reaction products.

Each member of a sequence-terminating non-Watson-Crick-pairing nucleotide pair should be labeled differently, such as having a different fluorophore, so that each member of a sequence-terminating non-Watson-Crick-pairing nucleotide pair can be detected, distinguished and measured separately. Further, each member of a labeled sequence-terminating non-Watson-Crick-pairing nucleotide pair, such as ddATP and ddCTP, can have differently labeled subsets for each fragment producing reaction performed, such as x1ddA, x2ddA . . . xnddA and y1ddC, y2ddC . . . ynddC, respectively, where x1, x2, . . . xn and y1, y2, . . . yn each represents different labels conjugated to the respective ddNTP, to allow further identification of the reaction products. Suitable labels include fluorescein, rhodamine 110, rhodamine 6G and carboxyrhodamine, among other labels. Suitable labeled sequence-terminating non-Watson-Crick-pairing nucleotides are commercially available from Amersham Pharmacia Biotech, Inc. and Perkin-Elmer Corporation, among other suppliers.

Further, the non-sequence-terminating nucleotides used in the reaction mixture can similarly be labeled to identify the reaction mixture which produced reaction products. This is accomplished by labeling all labeled non-sequence-terminating nucleotides used in a single fragment producing reaction with the same label, while labeling all labeled non-sequence-terminating nucleotides used in a different fragment producing reaction with a different distinguishable label. When used, labeled non-sequence-terminating nucleotides constitute only a fraction of the total amount of non-sequence-terminating nucleotides. When used, labeled non-sequence-terminating nucleotides are preferably present at a ratio of about 1% to 10% of the concentration of unlabeled non-sequence-terminating nucleotides. In a preferred embodiment, the non-sequence-terminating nucleotides are fluorescently labeled.

This reaction mixture is added to a suitable reaction vessel, such as 0.2 ml or 0.5 ml tubes or in the wells of a 96-well thermocycling reaction plate. Using this method, multiple polynucleotides can be analyzed simultaneously in the same physical location either by having pooled sample in the original template producing amplification reaction, or by pooling templates produced by the template producing amplification reactions. When multiple polynucleotides are being simultaneously analyzed by either option, the reaction mixture includes templates that are specific for each polynucleotide. Obviously, however, two polynucleotides can also be analyzed in separate physical locations simultaneously, to save time. Each reaction is then overlaid with an evaporation barrier, such as mineral oil or paraffin wax beads, and the reaction mixtures are cycled over suitable temperature ranges for suitable times.

Once the reaction mixture is placed in the appropriate vessel, the fragment producing reaction is accomplished according to techniques known to those with skill in the art, such as by standard PCR techniques using temperature cycling. This fragment producing reaction produces a set of labeled reaction products comprising a family of labeled complementary DNA strands terminated at every location beyond the primer by a sequence-terminating non-Watson-Crick-pairing nucleotide at the 3′ end where one of the nucleotides in the template strands contains a base corresponding to one of the sequence-terminating non-Watson-Crick-pairing nucleotide pairs.

By way of example only, typical times and temperatures required to accomplish the cycling conditions are a temperature over the range of 90° C. to 98° C. for a period of 10 seconds to 2 minutes for melting the template strands; a temperature range of 40° C. to 60° C. for an interval ranging from 1 second to 60 seconds to anneal the primers to their respective target strands; and a temperature range of 50° C. to 75° C. for an interval ranging from 30 seconds to 10 minutes to extend the primers by the action of the DNA polymerase. These cycles are repeated a sufficient number of times, generally between about 10 and 60 times, to obtain sufficient quantities of detectable labeled reaction products. In a preferred embodiment, the fragment producing reaction is performed using 25 cycles at 95° C. for 30 seconds, 50° C. for 5 seconds and 60° C. for 4 minutes. However, as will be understood by those with skill in the art with reference to this disclosure, the optimum times and temperatures will depend on the primer lengths, primer sequence, polynucleotide sequence being analyzed and the DNA polymerase utilized.

6) Analysis of Reaction Products:

After production of the family of labeled, linear polynucleotide fragments from both strands of the template, these labeled reaction products from the first polynucleotide are identified using the labels and the identity is compared to a known reference sequence or compared with the labeled reaction products produced from a second polynucleotide to determine the sequence variation between the first polynucleotide and the reference sequence or between the first polynucleotide and the second polynucleotide. This is accomplished as follows.

First, preferably, the labeled reaction products are purified from the other reaction mixture components by methods well known to those in the art, such as by ethanol precipitation. The purified labeled reaction products are then analyzed by an appropriate process using an appropriate instrument. The processes and instruments used for such an analysis must be capable of detecting and discriminating between the labels utilized in the fragment producing reaction method and must be capable of discriminating or resolving a single base difference between strands of single stranded DNA of different lengths.

For example, the purified labeled reaction products can be combined with suitable loading reagents and then analyzed using denaturing electrophoresis under conditions similar to those for standard polynucleotide sequencing. In summary, the reaction products are dissolved in water or other suitable buffer and are mixed with formamide. Then, they are denatured by heating at 95° C. for about 1 to 5 minutes and rapidly cooled at 4° C. Next, the denatured reaction products are loaded onto an appropriate instrument and analyzed using denaturing polyacrylamide electrophoresis or denaturing capillary electrophoresis or other suitable method where the instrument used is capable of detecting and distinguishing the labels on the reaction products. The separation matrix used for the electrophoresis must be capable of single base resolution for single stranded or denatured DNA. Suitable instrumentation is commercially available from Amersham Pharmacia Biotech, Inc., LiCor, Inc., Lincoln, Nebr. US and Perkin-Elmer Corporation, among other sources. Additionally, suitable custom-made instruments are also available, such as the SCAFUD from the Marshfield Institute, Marshfield, Wis. US. Both types of instruments have software for the analysis of the patterns produced by the detection of the fluorescent reaction products and for comparing the resulting data for each sample undergoing detection and analysis.

Once the labeled reaction products are analyzed, they are compared to a reference sequence or to similar reaction products from a second polynucleotide analyzed and the variations between the first polynucleotide and a reference sequence or between the first polynucleotide and the second analyzed polynucleotide can be determined. Additionally, the results of multiple analyses, and the sources and phenotypes of the samples can be compiled into data bases for additional analysis and correlation. Further, more than two polynucleotide sequences can be simultaneously analyzed using this method in a single reaction mixture, as will be understood by those with skill in the art with reference to this disclosure.

7) Interpretation of Labels Incorporated into Reaction Products:

The preferred modes of detection of the labeled reaction products produced by the present method detect and discriminate between the labels used in the method. The labels serve two different functions.

First, source-identifying labels are used to identify the source of the sequences represented by the reaction products by incorporating different, distinguishably labeled primers or labeled non-sequence-terminating nucleotides, or both, into the reaction products, where the same label is incorporated into reaction products derived from a single source or pool. Identifying the signal from these labels then allows determination of the source or pool from which the reaction product sequences were derived.

Secondly, base-identifying labels, which are different labels from the source-identifying labels, are used to identify the terminal base on a reaction product by incorporating different, distinguishably labeled sequence-terminating non-Watson-Crick-pairing nucleotides into the reaction products.

The uses of these two types of labels will be better understood by reference to the following examples. In the first example, the forward primer used in the fragment producing reaction has a red label (R) and the reverse primer used in the fragment producing reaction has a blue label (B). Further, the ddGTP member of the pair of sequence-terminating non-Watson-Crick-pairing nucleotides has a green label (G), and the ddTTP member of the pair of sequence-terminating non-Watson-Crick-pairing nucleotides has a yellow label (Y). In addition, a portion of the non-sequence-terminating nucleotides dCTPs have orange labels (O) for the fragment producing reaction containing templates from a first sample, and purple labels (P) for the fragment producing reaction containing templates from a second sample. Table I gives the expected results of the two fragment producing reactions and shows the distribution of labeled reaction products expected in this example.

TABLE I

| First Sample | | | | Second Sample | | | |
|---|---|---|---|---|---|---|---|
| dCTP | | Terminat | Reaction | dCTP | | Terminat | Reaction |
| Sample | Primer and | or and | Product | Sample | Primer and | or and | Product |
| Color | Color | Color | Colors | Color | Color | Color | Colors |
| O | Forward-R | ddGTP-G | O, R, G | P | Forward-R | ddGTP-G | P, R, G |
| O | Forward-R | ddTTP-Y | O, R, Y | P | Forward-R | ddTTP-Y | P, R, Y |
| O | Reverse-B | ddGTP-G | O, B, G | P | Reverse-B | ddGTP-G | P, B, G |
| O | Reverse-B | ddTTP-Y | O, B, Y | P | Reverse-B | ddTTP-Y | P, B, Y |

Thus, as can be appreciated from the above example, each reaction product can be identified as to its sample source, template strand and terminating base, while the location of the terminal base can be identified from the analysis of the length of the reaction products in combination with knowledge of the length of the template strand. In the above example, peaks with the colors orange, red and green within them arise from reaction products from the first sample because they contain orange, are from the forward primer containing template strands because they contain red, and are each terminated by base G because they contain green.

By considering the labels of the reaction products generating each peak and their relative positions from one another, a sequence for both the forward and reverse strands of the template can be determined. The sample from which the reaction products derived can be identified by their label and the sequence variations between a polynucleotide from a first sample and a polynucleotide from a second sample can be determined. Further, by analyzing relative intensities of peaks generated from the labeled reaction products from the two samples, an estimate of the relative frequency of the occurrence of the variation can be determined.

In the second example, the location of a polynucleotide variation on a single allele or on two alleles is determined. For this purpose, the fragment producing reaction is performed with entirely unlabeled dNTPs, but the forward primer used in the fragment producing reaction has a red label (R) and the reverse primer used in the fragment producing reaction has a blue label (B). Further, the ddGTP member of the pair of sequence-terminating non-Watson-Crick-pairing nucleotides has a green label (G), and the ddTTP member of the pair of sequence-terminating non-Watson-Crick-pairing nucleotides has a yellow label (Y). Table II gives the expected results and shows the distribution of labeled reaction products expected in this example.

TABLE II

| First Allele | | | Second Allele | | |
|---|---|---|---|---|---|
| | Term- | Reaction | | Term- | Reaction |
| Primer | inator | Products | Primer | inator | Product |
| and Color | and Color | Colors | and Color | and Color | Colors |
| Forward-R | ddGTP-G | R, G | Forward-R | ddGTP-G | R, G |
| Forward-R | ddTTP-Y | R, Y | Forward-R | ddTTP-Y | R, Y |
| Reverse-B | ddGTP-G | B, G | Reverse-B | ddGTP-G | B, G |
| Reverse-B | ddTTP-Y | B, Y | Reverse-B | ddTTP-Y | B, Y |

By reference to the known sequence, the peaks from the various reaction products can be determined to derive from either the forward or reverse strands. Then, a comparison of the resulting products arising from forward and reverse strands and their relative intensities and color allow a determination to be made as to whether the variation is present on one allele or two alleles.

### EXAMPLE I

### Using the Present Method to Locate and Identify an SNP from a Single DNA Sample from an Individual

The present method was used to determine the location and identity of two different single nucleotide polymorphisms in a region of DNA containing both the human growth hormone transcriptional activator (GHDTA) and the human growth hormone (GH1) genes. The method was performed separately on DNA from two different individuals. One individual was homozygous A at both loci 1 and 2. The other individual was homozygous G at loci 1 and homozygous T at loci 2. The method was performed as follows.

First, 2.7 kb templates spanning the region containing the GHDTA and GH 1 genes from each individual were separately prepared using PCR by standard methods. Then, fragment producing reactions were performed. The reaction mixtures contained fluorescent labeled 2′-3′ dideoxynucleotide triphosphates sequence-terminating non-Watson-Crick-pairing nucleotide pairs. Two reactions were performed on each sample. One reaction was performed using the pair ddATP:ddCTP (the “A/C reaction”) and another reaction was performed using the pair ddGTP:ddTTP (the “G/T reaction”).

Each reaction mixture contained components from an Amersham ThermoSequenase™ Dye Terminator Cycle Sequencing Core Kit according to the manufacturer's instructions, which comprised 1/10 the amount of the following components: 20 μl of 5× reaction buffer, 10 μl of dNTP mix, 20 μl deionized water, 10 μl of ThermoSequenase™, 120-150 ng of template, and 20 pMoles each of forward and reverse primers which spanned a 272 base pair sequence of the template between the primers' 5′ ends. The A/C reactions also contained 1 μl of rhodamine 6G labeled ddATP and 1 μl of ROX labeled ddCTP. The G/T reactions also contained 1 μl of rhodamine 110 labeled ddGTP and 1 μl of TAMRA labeled ddTTP.

A wax bead overlay was used to prevent evaporation during thermocycling. Cycles used in the fragment producing reaction consisted of an initial denaturation of 3.5 minutes at 96° C., an annealing of 15 seconds at 50° C., and an extension of 4 minutes at 60° C. Then, thirty additional cycles were performed consisting of 30 seconds at 96° C., 15 seconds at 50° C. and 4 minutes at 60° C. with a final extension of 10 minutes at 60° C.

Following cycling, the reaction mixture was chilled to 4° C. The wax overlay was removed and the reaction products were transferred to 1.5 ml tubes. Then, the DNA was precipitated by addition of 2 μl of 3 M sodium acetate (pH 5.2) and 68 μl of −20° C., 100% ethanol. The tubes were chilled to −20° C. for 10 minutes and then centrifuged for 5 minutes at 13,500×g.

Next, the ethanol was aspirated from the pellets and the pellets were washed with 300 μl of −20° C., 80% ethanol and centrifuged for 5 minutes at 13,500×g. The ethanol was aspirated and the pellets were briefly dried, then resuspended in 4 μl of deionized water. For the A/C and G/T sets, 2 μl of an internal standard MapMarker™ 400 (BioVentures, Inc., Murfreesboro, Tenn.) labeled with TAMRA or ROX was added, respectively. The samples were vortexed and then heated for 10 minutes at 37° C. to completely dissolve the pellets. The samples were briefly centrifuged to bring reaction products to the bottom of the tubes.

2 μl of each sample containing the reaction products was added to 10 μl of deionized formamide in 0.5 ml analysis tubes and capped with septa. The tubes were vortexed and briefly centrifuged. Then, the samples were denatured for 5 minutes at 95° C. and quickly chilled to 4° C.

Next, the reaction products were analyzed on an ABI PRISM™ 310 Genetic Analyzer from Perkin-Elmer Corporation using a 41cm uncoated column and POP 4 gel. The run module for the analyses comprised electrokinetic injection at 5 kV for 30 seconds, and electrophoresis at 15 kV for 24 minutes at 60° C. using appropriate spectral CCD modules for the dye sets. These conditions were utilized to resolve the fluorescently labeled reaction products. Data was processed using GeneScan7 analysis software from Perkin-Elmer Corporation, according to the manufacturer's instructions. For the A/C reactions, the channels corresponding to green (ddA Rhodamine 6G) and red (ddC ROX) were utilized for sample data, and the yellow (TAMRA) channel was utilized for the internal standard. For the G/T reactions, the blue, (ddG Rhodamine 110) and the yellow ddTTP (TAMRA) channels were utilized for sample data, and the red (ROX) channel was utilized for the internal standard.

The results obtained for each reaction were compared to the known DNA sequence for each of the individuals in the region flanked by the primers, and comparison demonstrated the proper location and identity of the SNPs. This demonstrates that the present method can be used to locate and identify a plurality of SNPs from a DNA sample from an individual.

### EXAMPLE II

### Using the Present Method to Locate and Identify a SNP from Pooled Template Mixtures and from Pooled Genomic DNA Samples

The present method was further used to locate and identify SNPs in mixtures of pooled templates, and in mixtures of pooled genomic DNA. First, mixtures of pooled 2.7 kb templates, each obtained as disclosed in Example I, were made using 150 ng/μl total DNA in the following template ratios: 1:0; 40:1; 20:1; 10:1; 1:1; 1:10; 1:20; 1:40; 0:1. Each of these pooled template mixtures was subjected to the present method as further disclosed in Example I. One reaction was performed using a ddATP:ddCTP sequence-terminating non-Watson-Crick-pairing nucleotide pair, and another reaction was performed using a ddGTP:ddTTP terminator pair. The reaction products were analyzed as in Example I.

The results demonstrated that the location and identity of the SNPs were determined by the present method even though the reaction mixtures contained pooled templates, and even when the templates were diluted as much as 1 in 40 with templates having the other alleles. Further, the relative intensities of peaks corresponding to each allele accurately represented the proportion of each allele in the reaction mixtures. This indicates that the frequency of an SNP in a pooled template mixture can be determined using the present method.

Second, mixtures of genomic DNAs from the same two individuals in Example I with different SNP genotypes were pooled in ratios of 1:0; 40:1; 20:1; 10:1; 1:1; 1:10; 1:20; 1:40; 0:1. This pooled genomic DNA was then used to obtain 2.7 kb templates. 120 ng total aliquots of the templates were purified and processed according to the present method as disclosed in Example I but using primers and using ddGTP:ddTTP terminator pairs, all of which were fluorescently tagged with different, distinctly identifiable fluorochromes.

The results produced distinctly identifiable patterns for each of the two templates. Two color tagged fragments appeared and their signal intensities vary with the proportion of the SNP found in the pooled mixture. That is, as the proportion of SNP1 (G) and SNP2 (T) alleles or the proportion of SNP1(A) and SNP2(A) increased or decreased, the signals associated the terminators on the corresponding fragments also similarly increased or decreased.

In contrast to uncolored ddF patterns produced by radiolabelling, this example demonstrates that patterns resulting from the present method can easily locate and identify different SNPs because the terminators were tagged with different fluorochromes which could be selectively identified by their color differences. Further, the reaction products resulting from SNPs were easily identified even when the templates were pooled or when pools of genomic DNA were used to produce pooled templates containing the SNP, and when the templates containing the SNP were diluted to as much as 1:40 with templates that did not contain the SNP.

### EXAMPLE III

### Using the Present Method to Locate and Identify Sequence Differences Simultaneously Between Multiple Polynucleotides Using Sequence-Terminating Non-Watson-Crick-Pairing Nucleotide Alpha Thio Analogs

The present method was further used to locate and identify sequence differences simultaneously between multiple polynucleotides using sequence-terminating non-Watson-Crick-pairing nucleotide alpha thio analogs as follows. First, a set of highly homologous DNAs was selected comprising pGEM vectors 3Z (+), 5Z (+), 7Z (+) and 11Z (+) obtained from Promega Corp. These vectors contained identical sequences throughout their entire closed circular length with the exception of the composition of their respective multiple cloning sites. A pair of PCR primers was designed to span the multiple cloning sites, having the multiple cloning site located at the approximate mid portion of each of the respective PCR amplicons obtained from the four respective vectors according to techniques known to those with skill in the art. The respective amplicon product sizes were 1003 bp for 3Z, 1060 bp for 5Z, 1057 bp for 7Z and 1027 bp for 11Z.

Individual PCR amplifications were performed using 50 pMoles of each primer and 100 ng of each of the pGEMs using 2.5 units of TAQ polymerase (Promega) in a total volume of 100 μl supplemented with 200 μmolar of each of the four dNTPs, as non-sequence-terminating nucleotides, in 1×PCR buffer (Promega) using 0.5 ml PCR tubes (Perkin Elmer Corporation) overlain with 20 μl mineral oil (Sigma-Aldrich) on a thermocycler (MJ Research, Boston, Mass. US) using cycles consisting of a first denaturation at 95° C. for 5 minutes followed by an annealing step of 58° C. for 30 seconds, followed by an extension step of 1 minute 30 seconds at 72° C. This set of cycles was repeated for a total of 30 cycles with the denaturation time at 95° C. reduced to 1 minute for the additional cycles. The final extension was for 8 minutes at 72° C.

Following amplification the correct product sizes were accessed by electrophoresis of 2 μl of PCR product for each reaction with size comparison to a DNA molecular weight marker on a precast 1% agarose gel (BMA Corp, Rockland, Me. US) containing ethidium bromide and photographing the stained gel on a transilluminator. The correct products were obtained for each pGEM. To further substantiate that the correct products were obtained for each pGEM, the PCR products were sequenced using the same primer individually as used for the amplification to obtain forward and reverse sequences of the PCR amplicons employing an ABI BigDye terminator cycle sequencing kit (ABI, Foster City, Calif. US) and following the manufacturer's protocol. The sequences obtained for each pGEM amplicon was in 100% agreement with the reported sequences for each of the pGEM vectors for the segment spanned by the PCR primers. The four pGEM PCR products were cleaned by standard ethanol precipitation and the recovered DNA was brought to 26.6 ng/μl based on A260 measurements in 0.1×TE buffer. The products were stored at −20° C. and thawed at room temperature for each further use.

Variations between the four pGEMs at their multiple cloning sites was determined by the present method using the reaction conditions described below. Primers were selected which nest within the approximate 1 KB amplicon of each of the pGEMs according to techniques known to those with skill in the art. Primers were synthesized using standard phosphoramidite chemistry and were obtained in unlabeled and labeled forms from Research Genetics, Huntsville, Ala. US. The forward and reverse primers were each individually obtained labeled at their 5′ end with either fluoresceine, tetrachlorofluoresceine or hexachlorofluoresceine (FAM, TET or HEX) respectively. Each primer was brought into solution in 1×TE 2% acetontrile at 100 pMoles/μl as a working stock and were stored at −20° C. until thawed for use.

A set of five reactions was performed on each of the four pGEM PCR amplicons. The first reaction set contained the following components for each PCR amplicon from pGEM 3Z, 5Z, 7Z and 11Z: 200 μmolar each of dATP, dCTP, dGTP, dTTP as the non-sequence-terminating nucleotides; 1 × reaction buffer and 1.6 units of ThermoSequenase™ (Amersham Pharmacia Biotech); and 2 μmolar of each sequence-terminating non-Watson-Crick-pairing nucleotides ddATP and ddCTP (Roche Molecular Systems, Indianapolis, Ind. US); 53.2 ng of pGEM PCR amplicon; 1.0 pMoles of TET labeled forward primer and 2.0 pMoles HEX GVS reverse primer in a total volume of 10 μl in either 0.2 ml PCR tubes or 0.2 ml wells of a 96 well PCR plate (Perkin Elmer Corporation). The second reaction set contained the following components for each PCR amplicon from pGEM 3Z, 5Z, 7Z and 11Z: 200 μmolar each of dATP, dCTP, dGTP, dTTP as the non-sequence-terminating nucleotides; 1× reaction buffer and 1.6 units of ThermoSequenase™ (Amersham Pharmacia Biotech); and 2 μmolar of each sequence-terminating non-Watson-Crick-pairing nucleotides alpha thio ddATP and alpha thio ddCTP (Roche Molecular Systems); 53.2ng of pGEM PCR amplicon; 1.0 pMoles of TET labeled GVS forward primer and 2.0 pMoles HEX GVS reverse primer in a total volume of 10 μl in either 0.2 ml PCR tubes or 0.2 ml wells of a 96 well PCR plate (Perkin Elmer Corporation). The third, fourth and fifth reaction sets contained the same components as set forth above except that the reaction sets contained 2 μmolar concentration of each sequence-terminating non-Watson-Crick-pairing nucleotide in the final 10 μl reaction mixture as follows: in reaction set three, the sequence-terminating non-Watson-Crick-pairing nucleotides were 2 μmoles each of 3′ deoxy ATP and 3′ deoxy CTP; in reaction set four, the sequence-terminating non-Watson-Crick-pairing nucleotides were 3′ azido 2′ deoxy ATP and 3′ azido 2′ deoxy CTP (Trilink Biotechnologies); and in reaction set five, the sequence-terminating non-Watson-Crick-pairing nucleotides were 3′ amino 2′ deoxy ATP and 3′ amino 2′ deoxy CTP (Trilink Biotechnologies).

The assembled reaction components of each of the five reaction sets were then cycled on a Thermocycler (MJ Research) using an initial denaturation temperature of 95° C. for 5 minutes followed by an annealing temperature of 55° C. for 20 seconds, followed by an extension at 68° C. for 1 minute for a total of 30 cycles, with the denaturation temperature at 95° C. for 30 seconds for each of the remaining 29 cycles following the first cycle.

Following cycling, 1 μl of each individual reaction was combined with 0.5 μl of MapMarker® 30-650 bp and 12 μl of deionized formamide (American Bioanalytical, Natick, Mass. US) in 0.5 ml capillary electrophoresis tubes (Applied Biosystems, Foster City, Calif.) Followed by denaturation at 95° C. for 5 minutes and quick chilling to 4° C. Samples were then analyzed on an Applied Biosystems Prism 310 Genetic Analyzer using a standard capillary and POP 4 gel and the manufacturer's buffer. The analytical conditions were as set forth in examples I and II above. Following electrophoresis the sample traces were overlain for each set of reactions using the GeneScan software provided by the instrument manufacturer. The resulting traces, when displayed for the TET labeled forward strands, clearly indicated identical traces for the respective forward strands until the multiple cloning regions were encountered where the traces diverged from one another, indicating the location where the strands diverged in sequence identity for each of the respective pGEMs. When the dye channel corresponding to the HEX labeled reverse strands was displayed, the traces tracked identically until the location of the multiple cloning region for the respective pGEMs was encountered, where the traces diverged reflecting the divergence in sequence at the multiple cloning sites for the different pGEMs on their reverse strands.

It was noted that the resolution for the peaks produced by each of the separate reaction sets differed between the reaction sets but was highly similar within a given reaction set. Surprisingly, the reaction set which utilized the sequence-terminating non-Watson-Crick-pairing nucleotides alpha thio analogs had the best peak to peak resolution of the five different terminator sets, especially in those regions containing three or more contiguous homologous bases, such as three consecutive A's.

Although the present invention has been discussed in considerable detail with reference to certain preferred embodiments, other embodiments are possible. Therefore, the scope of the appended claims should not be limited to the description of preferred embodiments contained in this disclosure. All references cited herein are incorporated by reference to their entirety.

As used herein, the term “comprise” and variations of the term, such as “comprising” “comprises” and “comprise,” are not intended to exclude other additives, components, integers or steps.

What is claimed is:

1. A method for determining the presence, location or identity, or a combination of these, of the nucleotides in a first polynucleotide, or for determining the presence, location or identity, or a combination of these, of one or more than one nucleotide difference between a first polynucleotides and a second polynucleotide, comprising:
a) providing a sample of the first polynucleotide;
b) selecting a region of the first polynucleotide potentially containing the variation;
c) subjecting the selected region to a template producing amplification reaction to produce a first plurality of double stranded polynucleotide templates which includes the selected region;
d) selecting a region of the templates potentially containing the variation;
e) producing a first family of labeled, linear polynucleotide fragments from both strands of the templates simultaneously by a fragment producing reaction including,
i) a set of at least two primers comprising a first primer and a second primer,
ii) at least four types of non-sequence-terminating nucleotides, comprising at least two different sets of two Watson-Crick-pairing nucleotides or nucleotide analogs, and
iii) two types of sequence-terminating non-Watson-Crick-pairing nucleotides or nucleotide analogs, comprising a first terminator and a second terminator;
where one or more than one of the non-sequence-terminating nucleotides or the sequence-terminating non-Watson-Crick-pairing nucleotides is a nucleotide analog;
where the first primer and the second primer flank the selected region of the template strands;
where the first primer has a first primer label and the second primer has a second primer label;
where at least a portion of one of the types of non-sequence-terminating nucleotides is labeled with a first nucleotide label;
where the first terminator is labeled with a first terminator label and the second terminator is labeled with a second terminator label;
where each of the first primer label, the second primer label, the first nucleotide label, the first terminator label and the second terminator label are all distinguishable from each other;
where each of the first family of fragments are terminated by either the first terminator or the second terminator at the 3′ end of the fragment; and
where the first family of fragments includes one or more than one fragment terminating at each possible base, represented by the either the first terminator or the second terminator, of that portion of the selected region of both template strands flanked by a primer; and
f) determining the location and identity of the bases in the selected region of the first polynucleotide by detecting the first primer label, the second primer label, the first nucleotide label, the first terminator label and the second terminator label present in the fragments.

2. The method of claim 1, additionally comprising comparing the location and identity of the bases determined with the location and identity of bases from a second polynucleotide, thereby identifying the presence and identity of a variation in a nucleotide sequence between the selected region of the first polynucleotide and a corresponding region of the second polynucleotide, after determining the location and identity of the bases in the selected region of the first polynucleotide.

3. The method of claim 1, where the selected region of the first polynucleotide comprises a plurality of discontinuous sequences on the first polynucleotide.

4. The method of claim 1, where the template producing amplification reaction comprises subjecting the selected region to PCR.

5. The method of claim 1, where the template producing amplification reaction comprises subjecting the selected region to RT-PCR.

6. The method of claim 1, where the first plurality of double stranded polynucleotide templates comprises double stranded nucleic acid strands of between about 50 and 50,000 nucleotides per strand.

7. The method of claim 1, further comprising purifying the temples to remove other amplification reaction components after subjecting the selected region to a template producing amplification reaction.

8. The method of claim 1, where the fragment producing amplification reaction comprises subjecting the selected region to PCR.

9. The method of claim 1, where the fragment producing amplification reaction comprises subjecting the selected region to RT-PCR.

10. The method of claim 1, where the selected region of the template strands is between about 100 and 1000 nucleotides per strand.

11. The method of claim 1, where the at least four types of non-sequence-terminating nucleotides comprise dATP, dCTP, dGTP and dTTP.

12. The method of claim 1, where the at least four types of non-sequence-terminating nucleotides comprise dATP, dCTP, dGTP and dUTP.

13. The method of claim 1, where one or more than one of the at least four types of the non-sequence-terminating nucleotides comprise an alpha thio dNTP analog.

14. The method of claim 1, where two of the at least four types of the non-sequence-terminating nucleotides comprise an alpha thio dNTP analog, two of the at least four types of the non-sequence-terminating nucleotides comprise dNTPs, and the two sequence-terminating non-Watson-Crick-pairing nucleotides comprise ddNTPs corresponding to the two alpha thio phosphate dNTPs.

15. The method of claim 14, where the two alpha thio phosphate non-sequence-terminating nucleotides are present in an initial concentration of between about 10% and 50% of that of the initial concentration of the two dNTP non-sequence-terminating nucleotides.

16. The method of claim 1, where one or more than one of the two types of the sequence-terminating non-Watson-Crick-pairing nucleotides comprises an alpha thio dNTP analog.

17. The method of claim 1, where one or more than one of the two types of the sequence-terminating non-Watson-Crick-pairing nucleotides is a 2′ deoxnucleotide triphosphates analog having an extension blocking moiety at the 3′ position.

18. The method of claim 17, where the extension blocking moiety is selected from the group consisting of an azide moiety, an amino moiety, a deoxy moiety, a fluoro moiety and a methoxy moiety.

19. The method of claim 1, where one or more than one of the sequence-terminating non-Watson-Crick-pairing nucleotides has an acyclo analog of a nucleotide sugar moiety.

20. The method of claim 1, where the first terminator comprises a pyrimidine nucleotide and where the second terminator comprises a purine nucleotide.

21. The method of claim 1, where the first terminator and the second terminator are selected from the group consisting of ddATP:ddCTP, ddATP:ddGTP, ddCTP:ddTTP, ddGTP:ddTTP, ddCTP:ddUTP and ddGTP:ddUTP and one of the foregoing pairs where one or both members of the pair is a nucleotide analog.

22. The method of claim 1, where one or more than one of the first primer label, the second primer label, the first nucleotide label, the first terminator label and the second terminator label are selected from the group consisting of fluorescent labels, fluorescent energy transfer labels, luminescent labels, chemiluminescent labels, phosphorescent labels and photoluminescent labels.

23. The method of claim 1, where the portion of one of the types of non-sequence-terminating nucleotides that is labeled with a first nucleotide label comprises between about 1% and about 10% of the total concentration of unlabeled nucleotide triphosphates.

24. The method of claim 1, further comprising purifying the labeled reaction products from the fragment producing reaction before determining the location and identity of the bases in the selected region of the first polynucleotide.

25. The method of claim 2, where the sequence of the corresponding region of the second polynucleotide is determined by:
a) providing a sample of the second polynucleotide;
b) selecting a region of the second polynucleotide which corresponds to the region of the first polynucleotide potentially containing the variation;
c) subjecting the corresponding region of the second polynucleotide to a template producing amplification reaction to produce a second plurality of double stranded polynucleotide templates which includes the corresponding region;
d) producing a second family of labeled, linear polynucleotide fragments from both strands of the template simultaneously by a fragment producing reaction including,
i) a set of at least two primers comprising a third primer and a fourth primer,
ii) at least four types of non-sequence-terminating nucleotides, comprising at least two different sets of two Watson-Crick-pairing nucleotides or nucleotide analogs, and
iii) two types of sequence-terminating non-Watson-Crick-pairing nucleotides or nucleotide analogs, comprising a third terminator and a fourth terminator;
where the third primer and the fourth primer flank the selected region of the template strands;
where each of the second family of fragments are terminated by either the third terminator or the fourth terminator at the 3′ end of the fragment; and
where the second family of fragments includes one or more than one fragment terminating at each possible base, represented by the either the third terminator or the fourth terminator, of that portion of the selected region of both template strands flanked by a primer;
e) determining the location and identity of at least some of the bases in the corresponding region of the second polynucleotide.

26. The method of claim 25, where the location and identity of the bases of the corresponding region of the second polynucleotide is determined simultaneously with determining the location and identity of the bases in the selected region of the first polynucleotide.

27. The method of claim 25, where producing the first family of labeled, linear polynucleotide fragments and producing the second family of labeled, linear polynucleotide fragments is performed in one reaction.

28. The method of claim 25, where the third primer has a third primer label and the fourth primer has a fourth primer label, and where the third primer label and the fourth primer label are distinguishable from each other.

29. The method of claim 25, where at least a portion of one of the types of non-sequence-terminating nucleotides in the production of the second family of labeled, linear polynucleotide fragments is labeled with a second nucleotide label.

30. The method of claim 25, where the third terminator is labeled with a third terminator label and the fourth terminator is labeled with a fourth terminator label, and where the third terminator label and the fourth terminator label are distinguishable from each other.

31. The method of claim 25, where the third primer has a third primer label, the fourth primer has a fourth primer label and at least a portion of one of the types of non-sequence-terminating nucleotides in the production of the second family of labeled, linear polynucleotide fragments is labeled with a second nucleotide label, and where the third primer label, the fourth primer label and the second nucleotide label are all distinguishable from each other.

32. The method of claim 25, where the third primer has a third primer label, the fourth primer has a fourth primer label, the third terminator is labeled with a third terminator label and the fourth terminator is labeled with a fourth terminator label, and where the third primer label, the fourth primer label, the third terminator label and the fourth terminator label are all distinguishable from each other.

33. The method of claim 25, where at least a portion of one of the types of non-sequence-terminating nucleotides in the production of the second family of labeled, linear polynucleotide fragments is labeled with a second nucleotide label, where the third terminator is labeled with a third terminator label and the fourth terminator is labeled with a fourth terminator label, and where the second nucleotide label, the third terminator label and the fourth terminator label are all distinguishable from each other.

34. The method of claim 25, where the third primer has a third primer label, the fourth primer has a fourth primer label, at least a portion of one of the types of non-sequence-terminating nucleotides in the production of the second family of labeled, linear polynucleotide fragments is labeled with a second nucleotide label, the third terminator is labeled with a third terminator label and the fourth terminator is labeled with a fourth terminator label, and where the third primer label, the fourth primer label, the second nucleotide label, the third terminator label and the fourth terminator label are all distinguishable from each other.

35. A method for determining the presence, location or identity, or a combination of these, of the nucleotides in a first polynucleotide, or for determining the presence, location or identity, or a combination of these, of one or more than one nucleotide difference between a first polynucleotides and a second polynucleotide, comprising:
a) providing a sample of the first polynucleotide;
b) selecting a region of the first polynucleotide potentially containing the variation;
c) subjecting the selected region to a template producing amplification reaction to produce a first plurality of double stranded polynucleotide templates which includes the selected region;
d) selecting a region of the templates potentially containing the variation;
e) producing a first family of labeled, linear polynucleotide fragments from both strands of the templates simultaneously by a fragment producing reaction including,
i) a set of at least two primers comprising a first primer and a second primer,
ii) at least four types of non-sequence-terminating nucleotides, comprising at least two different sets of two Watson-Crick-pairing nucleotides or nucleotide analogs, and
iii) two types of sequence-terminating non-Watson-Crick-pairing nucleotides or nucleotide analogs, comprising a first terminator and a second terminator;
where one or more than one of the non-sequence-terminating nucleotides or the sequence-terminating non-Watson-Crick-pairing nucleotides is a nucleotide analog;
where the first primer and the second primer flank the selected region of the template strands;
where each of the first family of fragments are terminated by either the first terminator or the second terminator at the 3′ end of the fragment; and
where the first family of fragments includes one or more than one fragment terminating at each possible base, represented by the either the first terminator or the second terminator, of that portion of the selected region of both template strands flanked by a primer; and
f) determining the location and identity of the bases in the selected region.

36. The method of claim 35, additionally comprising comparing the location and identity of the bases determined with the location and identity of bases from a second polynucleotide, thereby identifying the presence and identity of a variation in a nucleotide sequence between the selected region of the first polynucleotide and a corresponding region of the second polynucleotide, after determining the location and identity of the bases in the selected region of the first polynucleotide.

37. The method of claim 35, where the first primer has a first primer label and the second primer has a second primer label, and where the first primer label and the second primer label are distinguishable from each other.

38. The method of claim 35, where at least a portion of one of the types of non-sequence-terminating nucleotides is labeled with a first nucleotide label.

39. The method of claim 35, where the first terminator is labeled with a first terminator label and the second terminator is labeled with a second terminator label, and where the first terminator label and the second terminator label are distinguishable from each other.

40. The method of claim 35, where the first primer has a first primer label, the second primer has a second primer label and at least a portion of one of the types of non-sequence-terminating nucleotides is labeled with a first nucleotide label, and where the first primer label, the second primer label and the first nucleotide label are all distinguishable from each other.

41. The method of claim 35, where the first primer has a first primer label, the second primer has a second primer label, the first terminator is labeled with a first terminator label and the second terminator is labeled with a second terminator label, and where the first primer label, the second primer label, the first terminator label and the second terminator label are all distinguishable from each other.

42. The method of claim 35, where at least a portion of one of the types of non-sequence-terminating nucleotides is labeled with a first nucleotide label, where the first terminator is labeled with a first terminator label and the second terminator is labeled with a second terminator label, and where the first nucleotide label, the first terminator label and the second terminator label are all distinguishable from each other.

43. The method of claim 35, where the first primer has a first primer label, the second primer has a second primer label, at least a portion of one of the types of non-sequence-terminating nucleotides is labeled with a first nucleotide label, the first terminator is labeled with a first terminator label and the second terminator is labeled with a second terminator label, and where the first primer label, the second primer label, the first nucleotide label, the first terminator label and the second terminator label are all distinguishable from each other.

44. The method of claim 35, where the selected region of the first polynucleotide comprises a plurality of discontinuous sequences on the first polynucleotide.

45. The method of claim 35, where the template producing amplification reaction comprises subjecting the selected region to PCR.

46. The method of claim 35, where the template producing amplification reaction comprises subjecting the selected region to RT-PCR.

47. The method of claim 35, where the first plurality of double stranded polynucleotide templates comprises double stranded nucleic acid strands of between about 50 and 50,000 nucleotides per strand.

48. The method of claim 35, further comprising purifying the temples to remove other amplification reaction components after subjecting the selected region to a template producing amplification reaction.

49. The method of claim 35, where the fragment producing amplification reaction comprises subjecting the selected region to PCR.

50. The method of claim 35, where the fragment producing amplification reaction comprises subjecting the selected region to RT-PCR.

51. The method of claim 35, where the selected region of the template strands is between about 100 and 1000 nucleotides per strand.

52. The method of claim 35 where the at least four types of non-sequence-terminating nucleotides comprise dATP, dCTP, dGTP and dTTP.

53. The method of claim 35 where the at least four types of non-sequence-terminating nucleotides comprise dATP, dCTP, dGTP and dUTP.

54. The method of claim 35 where one or more than one of the at least four types of the non-sequence-terminating nucleotides comprise an alpha thio dNTP analog.

55. The method of claim 35, where two of the at least four types of the non-sequence-terminating nucleotides comprise an alpha thio dNTP analog, two of the at least four types of the non-sequence-terminating nucleotides comprise dNTPs, and the two sequence-terminating non-Watson-Crick-pairing nucleotides comprise ddNTPs corresponding to the two alpha thio phosphate dNTPs.

56. The method of claim 55, where the two alpha thio phosphate non-sequence-terminating nucleotides are present in an initial concentration of between about 10% and 50% of that of the initial concentration of the two dNTP non-sequence-terminating nucleotides.

57. The method of claim 35 where one or more than one of the two types of the sequence-terminating non-Watson-Crick-pairing nucleotides comprises an alpha thio dNTP analog.

58. The method of claim 35 where one or more than one of the two types of the sequence-terminating non-Watson-Crick-pairing nucleotides is a 2′ deoxnucleotide triphosphates analog having an extension blocking moiety at the 3′ position.

59. The method of claim 58, where the extension blocking moiety is selected from the group consisting of an azide moiety, an amino moiety, a deoxy moiety, a fluoro moiety and a methoxy moiety.

60. The method of claim 35 where one or more than one of the sequence-terminating non-Watson-Crick-pairing nucleotides has an acyclo analog of a nucleotide sugar moiety.

61. The method of claim 35 where the first terminator comprises a pyrimidine nucleotide and where the second terminator comprises a purine nucleotide.

62. The method of claim 35 where the first terminator and the second terminator are selected from the group consisting of ddATP:ddCTP, ddATP:ddGTP, ddCTP:ddTTP, ddGTP:ddTTP, ddCTP:ddUTP, ddGTP:ddUTP and one of the foregoing pairs where one or both members of the pair is a nucleotide analog.

63. The method of claim 35, where one or more than one of the first primer label, the second primer label, the first nucleotide label, the first terminator label and the second terminator label are selected from the group consisting of fluorescent labels, fluorescent energy transfer labels, luminescent labels, chemiluminescent labels, phosphorescent labels and photoluminescent labels.

64. The method of claim 35, where the portion of one of the types of non-sequence-terminating nucleotides that is labeled with a first nucleotide label comprises between about 1% and about 10% of the total concentration of unlabeled non-sequence-terminating nucleotides.

65. The method of claim 35, further comprising purifying the labeled reaction products from the fragment producing reaction before determining the location and identity of the bases in the selected region of the first polynucleotide.

66. The method of claim 35, where one or more of the first primer, the second primer, a portion of one of the types of non-sequence-terminating nucleotides, the first terminator and the second terminator is labeled, and where determining the location and identity of the bases in the selected region of the first polynucleotide is accomplished by detecting the label or labels.

67. The method of claim 36, where the sequence of the corresponding region of the second polynucleotide is determined by:
a) providing a sample of the second polynucleotide;
b) selecting a region of the second polynucleotide which corresponds to the region of the first polynucleotide potentially containing the variation;
c) subjecting the corresponding region of the second polynucleotide to a template producing amplification reaction to produce a second plurality of double stranded polynucleotide templates which includes the corresponding region;
d) producing a second family of labeled, linear polynucleotide fragments from both strands of the template simultaneously by a fragment producing reaction including,
i) a set of at least two primers comprising a third primer and a fourth primer,
ii) at least four types of non-sequence-terminating nucleotides, comprising at least two different sets of two Watson-Crick-pairing nucleotides or nucleotide analogs, and
iii) two types of sequence-terminating non-Watson-Crick-pairing nucleotides or nucleotide analogs, comprising a third terminator and a fourth terminator;
where the third primer and the fourth primer flank the selected region of the template strands;
where each of the second family of fragments are terminated by either the third terminator or the fourth terminator at the 3′ end of the fragment; and
where the second family of fragments includes one or more than one fragment terminating at each possible base, represented by the either the third terminator or the fourth terminator, of that portion of the selected region of both template strands flanked by a primer;
e) determining the location and identity of at least some of the bases in the corresponding region of the second polynucleotide.

68. The method of claim 67, where the location and identity of the bases of the corresponding region of the second polynucleotide is determined simultaneously with determining the location and identity of the bases in the selected region of the first polynucleotide.

69. The method of claim 67, where producing the first family of labeled, linear polynucleotide fragments and producing the second family of labeled, linear polynucleotide fragments is performed in one reaction.

70. The method of claim 67, where the third primer has a third primer label and the fourth primer has a fourth primer label, and where the third primer label and the fourth primer label are distinguishable from each other.

71. The method of claim 67, where at least a portion of one of the types of non-sequence-terminating nucleotides in the production of the second family of labeled, linear polynucleotide fragments is labeled with a second nucleotide label.

72. The method of claim 67, where the third terminator is labeled with a third terminator label and the fourth terminator is labeled with a fourth terminator label, and where the third terminator label and the fourth terminator label are distinguishable from each other.

73. The method of claim 67, where the third primer has a third primer label, the fourth primer has a fourth primer label and at least a portion of one of the types of non-sequence-terminating nucleotides in the production of the second family of labeled, linear polynucleotide fragments is labeled with a second nucleotide label, and where the third primer label, the fourth primer label and the second nucleotide label are all distinguishable from each other.

74. The method of claim 67, where the third primer has a third primer label, the fourth primer has a fourth primer label, the third terminator is labeled with a third terminator label and the fourth terminator is labeled with a fourth terminator label, and where the third primer label, the fourth primer label, the third terminator label and the fourth terminator label are all distinguishable from each other.

75. The method of claim 67, where at least a portion of one of the types of non-sequence-terminating nucleotides in the production of the second family of labeled, linear polynucleotide fragments is labeled with a second nucleotide label, where the third terminator is labeled with a third terminator label and the fourth terminator is labeled with a fourth terminator label, and where the second nucleotide label, the third terminator label and the fourth terminator label are all distinguishable from each other.

76. The method of claim 67, where the first primer has a first primer label, the second primer has a second primer label, the third primer has a third primer label, the second primer has a second primer label, at least a portion of one of the types of non-sequence-terminating nucleotides in the production of the first family of labeled, linear polynucleotide fragments is labeled with a first nucleotide label, at least a portion of one of the types of non-sequence-terminating nucleotides in the production of the second family of labeled, linear polynucleotide fragments is labeled with a second nucleotide label, the first terminator is labeled with a first terminator label, the second terminator is labeled with a second terminator label, the third terminator is labeled with a third terminator label and the fourth terminator is labeled with a fourth terminator label, and where the first primer label, the second primer label, the third primer label, the fourth primer label, the first nucleotide label, the second nucleotide label, the first terminator label, the second terminator label, the third terminator label and the fourth terminator label are all distinguishable from each other.
